(19) United States
(12) Patent Application Publication
(10) Pub. No.: US 2016/0008163 A1
(43) Pub. Date: Jan. 14, 2016

(54) MINI CONDOM

(71) Applicant: YOLANDA DENEICE FRAZIER, Harrisburg, PA (US)

(72) Inventor: YOLANDA DENEICE FRAZIER, Harrisburg, PA (US)

(21) Appl. No.: 14/329,917

(22) Filed: Jul. 12, 2014

(51) Int. Cl. A61F 6/04

(52) U.S. Cl. CPC A61F 6/04; A61F 2006/047; A61F 2006/049

(57) ABSTRACT

One embodiment of the mini condom (1) having a substantially tubular unitary mini body (10) a nipple reservoir at the closed end (12), a ring(s) (2) and (3) at the opening of the condom, an adhesive ring (4), which has adhesive material on the inner circumference of the condom's wall above the ring at the opening, and an additional ring (13) above ring (3). Other embodiments are described as shown.

### BACKGROUND

1. Field

This application relates to condoms and, in particular a condom which allows for a pleasurable oral sex experience by exposing the shaft of the penis, while preventing the spread of bodily fluids and pregnancies.

2. Prior Art

Condoms are commonly used to prevent pregnancies, the exchange of bodily fluids, and the spread of STD's. They are normally made of latex or animal material and contain a single band at the open end of the condom and a nipple reservoir at the closed end. The average length of the traditional condom is 7.5″, and covers the shaft of the penis.

Originally condoms were unrolled and fitted over the penis covering the entire shaft of the penis, which makes it difficult for the performer of oral sex to perform freely. Many performers dislike the taste/texture of the condoms, which hinders them from performing with 100% efficiency. The performers often decide to forgo the condom, in order to perform the act, which is unsafe due to the spread of bodily fluids. The length of the condom's body prevents the “receiver” of oral sex from feeling the warmth of the human tongue and/or tongue jewelry on, along, and around the shaft of the penis. Also, the performer is unable to navigate the shaft skillfully and uninterrupted, thereby causing this method of protection to be avoided all together during oral sex. In addition, there are numerous complaints, by both participants, regarding the discomfort of wearing a condom during coitus. Also, when condoms are worn, they have the tendency to slip off, due to the condom becoming stretched out, or from the lack of holding support.

Thereafter, several types of condoms were designed to allow for a more pleasurable coitus experience and to prevent slippage. U.S. Pat. Nos. 6,983,751 to Osterberg (2006) and 6,308,708 to Strauss, et al (2001) show condoms with textured bodies, however the textured body of the condom is known to create more of an irritation for some during coitus, instead of providing the added pleasure it was designed to create. Users want a condom which is oral-sex friendly, and prevents the spread of bodily fluids while allowing users to “feel” each other, until now this condom did not exist.

U.S. Pat. No. 8,066,008 to Glenn (2011) discloses an increased protection against slippage, however the condom includes an elastic band which stretches around the testicles in order to prevent the condom from slipping, this band is an uncomfortable fit for males and not likely to encourage the use of condoms to prevent the spread of bodily fluids or pregnancy.

Several types of condoms were created in order to prevent condom from dislodging during coitus—for example, U.S. Pat. Nos. 4,354,494 (1982), 4,942,885 (1990), 5,121,755 (1992), 821,382 (1906), and 5,351,699 (1994), although they will keep the condom in position, these devices require specifically constructed condoms and consequently have not received significant acceptance. Currently, there are not any condoms on the market that benefits both the performer and male receiver of oral sex.

The majority of condoms heretofore known suffer from a number of disadvantages:

The length of condom prevents the performer from skillfully performing oral sex.

The length of the condom prevents the male shaft from exposure and experiencing the pleasure of the warmth of the human tongue and/or tongue jewelry.

The length of the condom prevents users from experiencing the maximum amount of pleasure that may be safely obtained, while providing protection.

Condoms slip off during coitus.

Many choose not to use a condom during oral sex.

### SUMMARY

In accordance with one embodiment of the mini condom comprising a unitary tubular body, approximately half the length of traditional condoms, having rings/bands strategically placed at the open end of condom and along the body (optional), and an adhesive ring(s) on the inner circumference of condom's body, which is sufficiently strong as to hold the condom in place and support the condom in position, preventing/reducing slippage. The adhesive is preferably a pressure sensitive adhesive, advantageously of a type that is readily removed from the penis and most preferably without leaving any significant residue, allowing for ease of removal. The shortened length of the condom allows for a more pleasurable oral-sex experience, than that of conventional condoms, while preventing the exchange of bodily fluids and pregnancies.

### DRAWINGS

### Figures

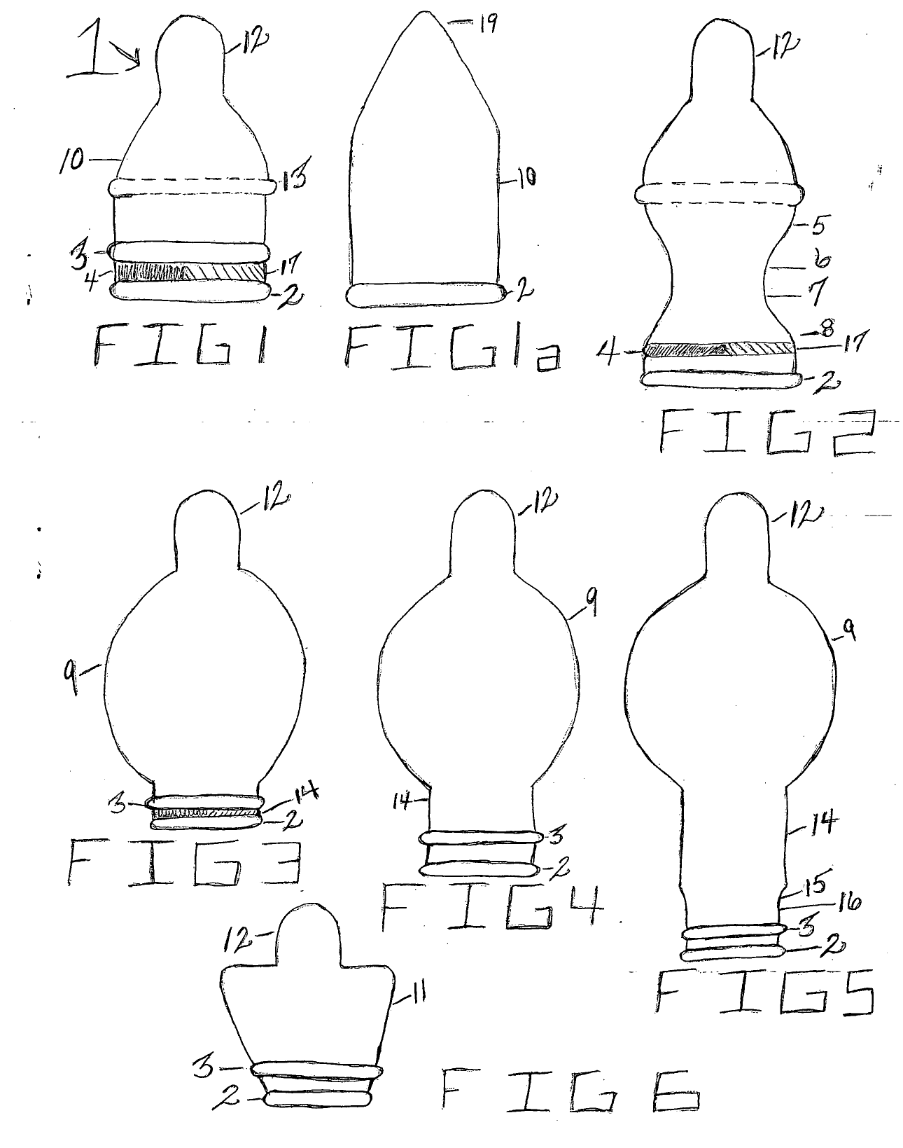

FIG. 1 shows the mini condom described and claimed in the aforesaid Patent Application; FIG. 1a shows the mini condom having a blunt closed end.

FIG. 2 shows the mini condom wherein a section of a reduced diameter occurs intermediate the closed and opened ends of a condom.

FIG. 3 shows the mini condom having a bulbous body section near its closed end and a ring at opened end and one ring above ring at open end.

FIG. 4 shows a similar mini condom having a bulbous body section near its closed end wherein a section of a reduced diameter occurs at the open end of the condom.

FIG. 5 shows a mini condom having a bulbous body section near its closed end and a section of reduced diameters occurs intermediate the closed and open ends of a condom.

FIG. 6 shows a mini condom having a diverging side walls from its open to closed ends.

### REFERENCE NUMERALS

mini condom—1
ring at condom opening—2
ring above first ring—3
adhesive rings (band)—4
section tapered sections: 5-8
bulbous body of condom—9
body of condom—10
diverging wall condom body—11
nipple reservoir at close end—12
additional rings—13
parallel side wall—14
inside diameter tapered at this points.—15&16
backing layer—17
additional adhesive rings—18
blunt reservoir at close end—19

### Operation—FIGS. 1-6

The open end of the condom is placed on the head of the penis and unrolled and applied in the same manner as the traditional condom. The condom is approximately 3.5 inches or less in total length from points 2 to 12. The rings formed on the condom 2 and 3 will have approximately equal outside/inside diameter, but ring of different outside/inside diameter may be used. The adhesive strip 4 is above the base ring 2 at the opening of the condom and has an adhesive material on the inside circumference of the wall 10 between rings 2 and 3, which allows rings 2 and 3 to secure the condom and prevent/reduce slippage, the thinness of the adhesive strip allows for easy removal, while still being effective. Additional embodiments are shown in FIGS. 2-6:

### DETAILED DESCRIPTION—FIGS. 1-6

One embodiment of the condom 1 FIG. 1, includes a reduced main body section 10, a ring 2 at its opening, a ring 3 above ring 2, and an adhesive ring 4 below ring 3, which can be a medical grade adhesive non-irritating to the users skin for adhering the condom to the users skin. As mentioned above the adhesive is preferably a pressure sensitive adhesive. The adhesive layer 4 is covered by backing layer 17 formed from a flexible material with a smooth surface in the case shown the backing layer covers the adhesive. The backing acts to protect the adhesive layer 4 from dirt and drying out. When using the respected condom 1, the user removes the backing layer 17 by separating, or peeling off, the backing layer 17 from the condom's inner wall and adhesive layer 4. When the backing layer 17 is peeled off, the adhesive layer 4 remains on the inner circumference of the condom wall 10, between rings 2 and 3 so that the condom 1 can be applied to adhere to the user's skin. As shown, an additional ring(s) 13 above ring 3 is/are optional this ring can be of smaller outside/inside diameter than that of the base ring, also to prevent slippage, there is a limit however, to the degree to which the diameter of the ring can be reduced without being as tight on the penis as to be objectionable. The closed end 12 has a nipple-shape for providing a reservoir for semen, in this example, but can be otherwise shaped, such as not including a reservoir but having a blunt end (19) 1a. By controlling the thickness of the rings non-slippage is enhanced, whereby a relatively thicker ring provides a tighter fit of the condom on penis.

The outside/inside diameters of the rings 2 and 3 are typically less than the outside/inside diameters of the body 10, respectively of condom so as to reduce slippage. The entire length of the condom is not greater than 3.7 inches may be less depending upon the embodiment. The pressure sensitive adhesive strip 4 is distributed on the inner circumference of the condom's wall.

FIG. 2, illustrates an embodiment of the invention wherein the condom has a first section 5 of constant diameter immediately below the closed end 12, and the diameter of the condom immediately below the first section is reduced or tapered between points 5 and 8 in a second section 6 to a smaller diameter which is retained in a third section 7, immediately below which the diameter gradually increases in a fourth section to the same diameter at the beginning of a fifth section 8 as the diameter of the first section 5. The fifth section 8 is of constant diameter proceeding down to the ring 2 at the open end, the inside diameter of ring 2 being the same as that of section 8. In one embodiment the diameter between points of sections 6 and 7 can be less than the inside diameter of the ring 2. An adhesive layer 4 is disposed on the inner circumference of condom's wall above ring 2.

Made in accordance with FIG. 2, the outside diameter at the ring 13 was 36 mm was the test subject. In a prototype condom, the end of the first section 5 nearest one open end was at 51 mm from the nipple-shaped closed end 12, the tapered second section 6 had a length of 13 mm, the third section 7 had a length of 10 mm, the tapered fourth section had a length of 13 mm, and the length of the fifth section 8 was 22 mm, for example. The condom itself typically has a length of 108+−10 mm. This, of course, is an example of one condom design, but other condom designs may use different dimensions.

FIG. 3, illustrates a third embodiment of the invention wherein the condom has a main bulbous body section 9, a ring 2 at open end, a ring 3 above ring 2, and an adhesive layer 4, which is disposed over a substantial area on the inner circumference of the condom's wall 14.

FIG. 4, illustrates a fourth embodiment of the invention, the condom has a bulbous body section 9 proximate the closed end, followed by substantially parallel side wall sections 14 to open end at ring 2. As in other embodiments, in addition to rings 2 and 3, different combinations of rings 13 and 4 may be used.

FIG. 5, illustrates a fifth embodiment, the condom has a bulbous body section 9 proximate closed end and a first section of constant diameter immediately below the closed end (14), and the diameter immediately below the first section is reduced or tapered between points 15 and 16 in the second section to the inside diameter of ring 3 and in the third section retains the diameter until the open end where ring 2 is located This diameter reduction along the material from the open end to a desired outside diameter. Ring 2 can be formed in the same manner as ring 3 as taught in the previously mentioned Ser. No. 821,382. Ring 13 is similarly formed. The friction of the section 15-16 of a reduced diameter adds to the friction provided by the ring 3, but because the condom is made of light material it does increase the sensation of tightness, but not in an objectionable manner. A double sided sticky adhesive pad 19 is used to secure condom.

FIG. 6, illustrates a sixth embodiment of the invention, the condom has successively and linearly increasing diameter from its open end at ring 2 to its closed end 12, producing diverging sidewalls 11 from the open to closed ends. In addition to rings 2 and 3, adhesive glue may be used in order to prevent/reduce slippage, different combinations of rings 2, 3, and the above mentioned adhesive methods may be used depending upon the desired condom configuration.

### Advantages

From the description above, a number of advantages of some embodiments of my condom become evident:

Due to the reduced coverage of the shaft oral sex is more pleasurable for both the performer and receiver

The shaft remains exposed allowing the male to reap the benefits of a warm tongue and/or tongue jewelry.

The performers of oral sex are able to navigate the shaft more skillfully.

The performers of oral sex are able to enjoy and complete the job w/o fear of tasting semen.

The reduced length of the condoms body will allow for actual contact between participants.

The rings allow the condom to remain in place.

The adhesive gives the extra support needed in order to prevent slippage.

The slight pressure from the rings, when strategically placed, below the head of the penis causes a more pleasurable orgasm for the male.

The exposed shaft of the male allows for a more pleasurable coitus experience for both participants.

### Conclusion, Ramifications, and Scope

Accordingly, the reader will see that the mini condoms of the various embodiments are a new and innovative way to enjoy oral sex for both the performer and receiver. By allowing the shaft of the penis to remain exposed, the receiver is able to feel the warmth of the human tongue/tongue jewelry and the performer is able to perform skillfully navigate the shaft, with the added bonus of not tasting semen. The condom is held in place by the strategically placed rings and adhesive strips. The exchange of bodily fluids is prevented and the performers of oral sex are able to do so skillfully, without the interference of the condom, its unpleasant taste/texture, or the concern of tasting semen.

Although the description above contains much specificity, these should not be construed as limiting the scope of the embodiments but merely providing illustrations of some of the presently preferred embodiments. For example, different combinations of specific features of the condom configurations of FIGS. 1 through 6 can be used to obtain other condom designs. Also, the bodies of the condoms can be selectively textured, flavored, and colored as is known in the art, for each of the various embodiments of the invention.

I claim:

1. A mini condom comprising:
a unitary substantially tubular mini body, the improvement wherein said condom's total length is reduced to extends slightly over the head of the penis, exposing the shaft;
an open end and an opposite closed end, including proceeding from said closed end to said open end, a first successive section;
said first section having a constant inside diameter.
a first ring formed about said open end; and a second ring having inside and outside diameters equal to, less than, or greater than those of said ring, thereby controlling the relative thickness of the rings.
an adhesive strip, wherein said layer is disposed over the inner circumference of condom's wall.
wherein said adhesive portion is covered by a release element prior to use.
parallel side walls continuously extending between said open and closed ends; and
at least a second ring formed between said open and closed ends, for enhancing non-slip properties of said condom, wherein the thickness of one of said first and second rings is made greater than the other for further enhancing the non-slip properties of said condom.

2. The mini condom of claim 1, further including a nipple-shaped reservoir for semen formed at the closed end.

3. The mini condom of claim 1, wherein said condom consists of material selected from the group consisting of latex rubber, animal skin, and a synthetic material.

4. A mini condom comprising of claim 1 wherein;
an unitary substantially tubular body mini condom having opposing closed and open ends, including proceeding from said closed end to said open end; first, second, and third successive sections.
said body further includes a second section having a gradual linear reduction in inside diameter from said first diameter to a second inside diameter at an intersection with one end of said third section;
said third section having said second inside diameter constantly between said second section and said open end;

5. A mini condom as set forth in claim 1, wherein another end of said third section is adjacent said open end.

6. A mini condom as set forth in claim 1, further comprising:
said mini condom further including a fourth section having one end integral with end of said third section, said fourth section having a diameter that gradually linearly increases from said second diameter to said first diameter; and
said mini condom further including a fifth section having one end integral with another end of said fourth section, said fifth section having said first diameter constantly between said fourth section and said open end.
a first ring at said open end; and
a second ring adjacent said first ring having inside and outside diameters equal to, less than, or greater than those of said first ring, thereby controlling the relative thickness of the rings.

7. A mini condom in accordance with claim 4, wherein said adhesive layer comprises pressure-sensitive adhesive.

8. A mini condom in accordance with claim 4, wherein said adhesive layer is disposed over a substantial area of said inner circumference of condom body.

9. A mini condom in accordance with claim 4, wherein said mini condom comprises a backing layer affixed to at least a portion of said adhesive layer.

10. A mini condom in accordance with claim 4, wherein said backing layer comprises coated paper.

11. The method of claim 1 wherein said adhesive portion is moisture activated glue.

12. The method of claim 1 wherein said adhesive securement is a double-sided sticky member that is provided separate from the condom before use, the method further comprising the step of applying the double-sided sticky member to the condom prior to the adhesively securing step.

13. The method of claim 1 wherein said adhesive securement is a flowable contact adhesive that is applicable to the penis, the condom, or both, the method further comprising the step of applying the flowable contact adhesive to the condom, the penis, or both, prior to the adhesively securing step.

14. The mini condom of claim 1 wherein:
a bulbous section having a first end integral with said closed end.
a first ring formed about said open end; and
at least a second ring formed below the first ring, for enhancing non-slip properties of said condom.

15. The mini condom of claim 7 wherein
an open end and an opposite closed end;
a bulbous section having a first end integral with said closed end, and a second end;
a short parallel walled section having a first end integral with said second end of said bulbous section, and a second end providing said open end;
a first ring formed about said open end; and
at least a second ring formed on said parallel walled section, for enhancing non-slip properties of said condom.

16. The condom of claim 7 wherein;
an open end and an opposite closed end;
a bulbous section having an opposing closed and open ends, including proceeding from said closed end to said open end, first, second, and third successive sections;
a first section having a constant first inside diameter;
said second section having a gradual linear reduction in inside diameter from said first diameter to a second inside diameter at an intersection with one end of said third section;
a first ring at said open end; and
a second ring adjacent said first ring having inside and outside diameters equal to, less than, or greater than those of said first ring, thereby controlling the relative thickness of the rings.

17. A mini condom as set forth in claim 7, wherein another end of said third section is adjacent said open end.

18. The mini condom in claim 1 wherein:
open end and an opposite closed end;
continuous linearly diverging side walls from said open end to said closed end;
a first ring formed about said open end; and
at least a second ring formed between said open and closed ends.

19. The mini condom of claim 11, further including:
a plurality of other rings formed along said body and spaced apart from one another and from said first and second rings.

20. A method for enjoying oral sex while preventing the exchange of bodily fluids.
a) Providing a mini body condom allowing the shaft to be exposed.
b) Allowing the tongue moves freely along the shaft.
Whereby said condom is not in the way of oral performance.
c) The method of claim 13 wherein said condom is composed of latex rubber, animal skin, and a synthetic material.
